(19) United States
(12) Patent Application Publication
(10) Pub. No.: US 2006/0269527 A1
(43) Pub. Date: Nov. 30, 2006

(54) ISOLATION OF CELLS FROM BONE

(75) Inventors: SUSAN KAYE NILSSON, East Bentleigh (AU); DAVID NORMAN HAYLOCK, Melbourne (AU)

Correspondence Address: BOZICEVIC, FIELD & FRANCIS LLP, 1900 UNIVERSITY AVENUE, SUITE 200, EAST PALO ALTO, CA, 94303, US

(73) Assignee: Australian Stem Cell Centre Limited

(21) Appl. No.: 11/420,336

(22) Filed: May 25, 2006

Related U.S. Application Data
(60) Provisional application No. 60/685,259, filed on May 26, 2005.

(51) Int. Cl. A61K 35/14; C12N 5/06; C12N 5/08

(52) U.S. Cl. 424/93.7; 435/372; 435/325; 435/354

(57) ABSTRACT

The present invention provides a method for isolating cells from bone(s) harvested from a mammal. The method comprising the steps of: (a) obtaining a bone(s) containing bone marrow from the mammal; (b) fragmentation of the bone(s); (c) washing the bone fragments produced from step (b) and recovery of dislodged cells; (d) treating the bone fragments with a composition to release cells adhered to the endosteal region of the bone; (e) harvesting the cells following treatment of the bone fragments according to step (d); and (f) optionally combining the cells from steps (c) and (e).

### CROSS-REFERENCE

This application claims the benefit of U.S. Provisional Application No. 60/685,259 filed May 26, 2005, which application is incorporated herein by reference.

### FIELD OF THE INVENTION

The present invention relates to methods for isolating cells from bone containing bone marrow. More particularly, it relates to methods for increasing the absolute number of hematopoietic stem and/or progenitor cells with improved qualities compared with hematopoietic stem and/or progenitor cells isolated by conventional prior art methods.

### BACKGROUND OF THE INVENTION

Hematopoietic stem cells (HSC) are the most primitive cells of the hematopoietic lineage, and have the ability to give rise to all cells of the hematopoietic lineage (including HSC). The differentiated progeny of HSC comprise the lymphoid, myeloid and erythroid lineages. The lymphoid lineage provides for the regulation of the immune system, the myeloid lineage monitors for the presence of foreign bodies in the blood stream and act as scavengers of such foreign bodies, and the erythroid lineage provides red blood cells which carry oxygen through the blood stream.

HSC are known to reside in the bone marrow, but their specific niche within the bone marrow microenvironment is not currently defined, presumably because HSC constitute only a small percentage of total cells in the bone marrow. Due to the rarity of HSC and the lack of a single, unique antigenic marker allowing their unambiguous identification in situ, the identification and purification of stem cells has been elusive.

Various surface markers used to characterise murine stem cells include the stem cell antigen (Sca-1), the tyrosine kinase receptor encoded by c-Kit, and Thy-1. Such cells are also characterised by their lack of surface expression of lineage specific markers (Lin−) including CD4, CD8, B220, Mac-1, Gr-1, CD5 and Ter119.

It has been demonstrated that a Sca-1+, Thy-llo population lacking demonstrable expression of lineage markers (Lin−) is significantly enriched in HSCs with long term repopulating ability. The Thy-lloSca-1+Lin− population, which comprises about 0.05% of total bone marrow cells, was able to differentiate into myelomonocytic, B and T cells with approximately unit efficiency and also able to completely reconstitute the natural killer (NK), erythrocyte and platelet lineages. HSC in normal bone marrow also express high levels of c-Kit.

In order to obtain HSCs it is necessary to isolate the rare pluripotent stem cells from other cells present in the bone marrow or other hematopoietic source. In general, harvesting of stem or progenitor cells from alternative sources in adequate amounts for therapeutic and research purposes is generally laborious, the sources are limited due to the nature of the harvesting procedures, and the yield is low.

Bone marrow cells may be obtained from a source of bone marrow eg. tibiae, femora, iliac crest and other bone cavities. Typically, for isolation of HSCs from bone marrow, an appropriate solution must be used to flush the bone marrow cavity. The solution is usually a balanced salt solution supplemented with fetal calf serum or other naturally occurring factors.

Optimisation of the yield of HSCs obtained from bone marrow requires that sufficient flushing force be used to ensure that HSCs, which reside within the bone marrow microenvironment are removed. Accordingly, the method of isolating HSCs from the bone marrow in this manner is likely to result in the yield and quality of HSCs varying considerably.

Accordingly, there is a need for an improved method for the isolation of murine HSCs and primitive progenitor cells from the bone marrow. This would assist in optimising the number of starting HSCs for research purposes.

### SUMMARY OF THE INVENTION

In a first aspect, the present invention provides a method for isolating cells from bone(s) harvested from a mammal, the method comprising the steps of:
(a) obtaining a bone(s) containing bone marrow from the mammal;
(b) fragmentation of the bone(s);
(c) washing the bone fragments produced from step (b) and recovery of dislodged cells;
(d) treating the bone fragments with a composition to release cells adhered to the endosteal region of the bone;
(e) harvesting the cells following treatment of the bone fragments according to step (d); and
f) optionally combining the cells from steps (c) and (e).

In a second aspect, the present invention provides a method for isolating cells from bone(s) harvested from a mammal, the method comprising the steps of:
(a) obtaining a bone(s) containing bone marrow from the mammal;
(b) flushing the cavity of the bone to remove central bone marrow;
(c) fragmentation of the bone(s);
(d) washing the bone fragments produced from step (c) and recovery of dislodged cells;
(e) treating the bone fragments with a composition to release cells adhered to the endosteal region of the bone;
(f) harvesting the cells following treatment of the bone fragments according to step (e); and
(g) optionally combining the cells from steps (d) and (f).

In a third aspect, the present invention provides a method for isolating hematopoietic stem cells and/or progenitor cells from bone(s) harvested from a non-human mammal, the method comprising the steps of:
(a) administering to at the least one mammal an agent used to eliminate proliferating cells;
(b) obtaining a bone(s) containing bone marrow from the mammal;
(c) fragmentation of the bone(s);
(d) washing the bone fragments produced from step (c) and recovery of dislodged cells;
(e) treating the bone fragments with a composition to release cells adhered to the endosteal region of the bone; and
(f) harvesting the cells following treatment of the bone fragments according to step (e); and
(g) optionally combining the cells from steps (d) and (f).

In a fourth aspect, the present invention provides a kit for isolating hematopoietic stem cells and/or progenitor cells from bone(s) harvested from a mammal, the kit comprising:
(a) a composition to release cells adhered to the endosteal region of the bone;
(b) one or more reagents for harvesting, washing and suspending the cells; and
(c) instructions for use of the reagents.

These and other objects, advantages, and features of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 shows the proportion of LSK cells within the starting bone marrow (SBM) and lineage negative (Lin−) fractions as well as the total number of LSK isolated using the three separative strategies. Values are the mean+std error of the mean (SEM) of at least 8 individual groups of animals.

FIG. 2 shows an analysis of the frequency and content of low-proliferative-potential colony-forming-cells (LPP-CFC) (A and B respectively) and high-proliferative-potential colony-forming-cells (HPP-CFC) (C and D respectively) in the central (flushed (left bar)) versus endosteal (ground (middle bar)) or enhanced(norm (right bar)) fractions. E, proliferation of LSK cells isolated from endosteal (gr) region versus central (fl) region after 6 days cultures in Cellgro plus 4 factor. Values are the mean+SEM of at least 3 dishes from 3 individual groups of animals.

FIG. 3 shows an analysis of whole bone marrow (BM) (A) and LKS (B) following the competitive transplant of various numbers of LSK isolated from the endosteal region (PTP) versus central region (57). Values are the mean±SEM of at least 5 animals per group.

FIG. 4 shows the ability of transplanted LSK isolated from the endosteal region to home to the BM and specifically the endosteal region compared to those isolated from the central region. Values are the mean±SEM of 2 individual donor populations for each fraction into between 2 and 4 recipient animals.

FIG. 5 shows spatial distribution of LSK 15 hr post transplant. Values are the mean±SEM of 3 individual animals.

### DETAILED DESCRIPTION OF THE INVENTION

Before the present methods and kits are described, it is to be understood that this invention is not limited to the particular methodology and reagents described, as such methods and reagents may, of course, vary. It is also to be understood that the terminology used herein is for the purpose of describing particular embodiments only, and is not intended to limit the scope of the present invention which will be limited only by appended claims.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It must be noted that as used herein and in the appended claims, the singular forms “a,” “and,” and “the” include plural referents unless the context clearly dictates otherwise.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

### Definitions

The term “hematopoietic stem cell (HSC)” as used herein means a stem cell, also referred to as a multipotential or pluripotential stem cell having (1) the ability to give rise to progeny in all defined hematopoietic lineages, and (2) stem cells capable of fully reconstituting a seriously immunocompromised host in all blood cell types and their progeny, including the multipotential hematopoietic stem cell, by self renewal. The HSC is also intended to include stem cells identifiable by in vivo and in vitro assays such as long-term repopulating stem cells, spleen colony forming unit (CFU-S) and blast colony forming units (CFU-blast) which detects precursors which generate small undifferentiated colonies composed almost exclusively of undifferentiated blast cells which have a high replating efficiency.

The term “progenitor cells” as used herein is intended to refer to cells including the lympho-myeloid stem cell (LMSC) and the lymphoid and myeloid progenitor cells descended therefrom which are the precursors for the cells comprising the lymphoid and myeloid lineage respectively, it also refers to cells which are discernible by in vitro assay including mixed colony forming units (CFU-mixed) or CFU-GEMM which produce colonies containing erythroid cells, neutrophils, macrophages, eosinophils, and megakaryocytes.

The term “fragmentation” as used herein is meant that a substantial proportion of the bone is in fragments of a size range of from about 40 microns to about 1000 microns.

### Invention in General

The present Applicants have developed methods for improving the isolation of murine hematopoietic stem cells (HSCs) and primitive progenitor cells from bone containing bone marrow, in particular, the methods allow the efficient isolation of HSC and progenitor cells which are located in the endosteal region of the bone marrow. The Applicants have surprisingly found that enzymatic treatment of bone containing bone marrow provides a significantly greater yield, in terms of absolute numbers of HSCs and/or progenitor cells with improved qualities compared with prior art methods for the isolation of such cells. The methods of the invention are also suitable for isolating malignant cells that exist within the bone marrow or near the endosteal region.

In a first aspect, the present invention provides a method for isolating cells from bone(s) harvested from a mammal, the method comprising the steps of:
(a) obtaining a bone(s) containing bone marrow from the mammal;
(b) fragmentation of the bone(s);
(c) washing the bone fragments produced from step (b) and recovery of dislodged cells;
(d) treating the bone fragments with a composition to release cells adhered to the endosteal region of the bone;
(e) harvesting the cells following treatment of the bone fragments according to step (d); and
(f) optionally combining the cells from steps (c) and (e).

In a second aspect, the present invention provides a method for isolating cells from bone(s) harvested from a mammal, the method comprising the steps of:
(a) obtaining a bone(s) containing bone marrow from the mammal;
(b) flushing the cavity of the bone to remove central bone marrow;
(c) fragmentation of the bone(s);
(d) washing the bone fragments produced from step (c) and recovery of dislodged cells;
(e) treating the bone fragments with a composition to release cells adhered to the endosteal region of the bone;
(f) harvesting the cells following treatment of the bone fragments according to step (e); and
(g) optionally combining the cells from steps (d) and (f).

The methods described above are particularly effective at removing cells which are located near the endosteal region (in contrast to the central region) of the bone cavity. Adjacent to the endosteum are cells of the bone marrow. Cell types located in the endosteal region of the bone marrow include the primitive hematopoietic stem and progenitor cells. Furthermore, malignant cells are also known to reside in the endosteal region of bone. The method will increase the likelihood of isolating malignant cells that exist within the bone marrow or near the endosteal region. This is important because the bone is a frequent site for tumour metastasis and the ability to measure small amounts of tumour is of value in monitoring disease status/progression and response to treatment. At present, marrow micro-metastasis is assessed by microscopic examination of either a bone marrow aspirate or trephine section. The methods of the invention will enable malignant cells that might reside within the endosteal region to be released from trephine sections and thus increase the sensitivity of minimal residual disease detection.

In contrast, prior art methods for isolation of cells typically rely on flushing the bone marrow cavity with a buffer by means of a syringe. This generally only removes the central region of the bone marrow. Moreover, the method results in an inconsistent yield and quality of cells being obtained, particularly those of the hematopoietic stem and progenitor type as the efficiency of flushing can vary between one experiment to the next. Such flushing methods are also unlikely to enable the isolation and examination of malignant cells as described above.

Accordingly, in one embodiment, the cells according to the first and/or second aspect are hematopoietic stem and/or progenitor cells.

In another embodiment, the cells according to the first and/or second aspect are malignant cells. The method can be used to increase the recovery of malignant cells from a trephine section taken from a human. In this case the trephine section is used to assess for presence of bone marrow malignant cells. The section can be mechanically fragmented and subject to the same enzymatic process as for release of haemopoietic stem cells so as to release malignant cells. This will enhance the ability to detect low numbers of malignant cells attached to the bone surface or in the endosteal region.

The mammal may be a human or non-human mammal. Preferably, the non-human mammal is a mouse. Where the mammal is a human, then preferably, the harvested bone marrow cells are from a cadaveric source.

In order to optimise the purity of the cells harvested according to the methods of the invention, it will be evident to persons skilled in the art upon reading the present invention, that the bones when harvested from the mammal are preferably free of all muscle tissue. This will avoid muscle cells contaminating the harvested bone marrow cells. The muscles can be readily removed from the bone during dissection by cutting the tendons connecting the muscle to the bone.

It will be readily apparent to persons skilled in the art of the present invention that any bone containing bone marrow can be used in accordance with the methods of the invention. Where the mammal is a non-human mammal such as a mouse, then preferably the bone(s) containing bone marrow are selected from one or more of the group consisting of femur, tibia and iliac crest. Where the mammal is a human mammal, preferably the bone(s) containing bone marrow are selected from one or more of the group consisting of spine, rib, sternum, femur and iliac crest.

Since the proportion of HSCs and/or progenitor cells is considerably low in the bone marrow, in order to obtain a sufficient yield of HSCs and/or progenitor cells for further study in small non-human mammals, it may necessary to use the femurs harvested from multiple animals, e.g., at least 10-12 mice.

The method of flushing the bone cavity to remove the central bone marrow according to the second aspect of the invention is preferably conducted by using a syringe to flush a buffer such as phosphate buffered saline through the hollow cavity of the bone. For a non-human mammal such as a mouse, 26 gauge or 23 gauge needles are sufficient to flush the bone marrow from the bone(s).

The step of fragmenting the bone to produce bone fragments will be familiar to persons skilled in the art. Preferably, the bone(s) are fragmented by grinding them in a mechanical grinding device such as a mortar and pestle. Typically, the bone(s) are ground in the presence of a buffer such as phosphate buffered saline (PBS) containing heat inactivated serum which assists in maintaining the viability of the bone marrow cells. Alternatively, a mechanical device such as a polytron or a bone mill may be employed in order to fragment the bone. Alternatively, the bone(s) may be fragmented by chopping using a knife or other such device known in the art. It is to be understood that the method of fragmenting the bone(s) are not limited to those described above and that various other methods of fragmenting bone(s) will be familiar to persons skilled in the art and are considered to be within the scope of the present invention.

The fragmentation process results in a substantial proportion of the bone consisting of fragments of a size range of from about 40 microns to about 1000 microns.

Preferably the composition which releases cells adhered to the endosteal region of the bone comprises a collagenase and optionally an agent selected from the group consisting of a protease, a glycosidase and chelating agent and combinations thereof.

Preferably, the protease is dispase.

Preferably, the glycosidase is hyaluronidase.

Preferably, the chelating agent is ethylenediaminetetraacetic acid (EDTA).

Most preferably, the composition comprises a combination of 4 mg/ml dispase II and 3 mg/ml collagenase I.

Dispase, or neutral protease is a metalloenzyme produced by Bacillus polymyxa. It has been classified as an amino-endo peptidase. Dispase is typically supplied as a lyophilised product. Dispase is suitable for tissue disaggregation and subcultivation procedures since it does not damage cell membranes and also assists in preventing cell clumping. Dispase II is a more concentrated form of Dispase. Preferably, the concentration of Dispase II used according to the invention is between 0.5 to 10 mg/ml, even more preferably between 2-8 mg/ml, and most preferably between 3 and 5 mg/ml.

Collagenase I is purified from Clostridium histolyticum and is formulated in phosphate buffered saline with 20% fetal bovine serum. One of its known uses is for the disruption of embryoid bodies derived from embryonic stem cells. Preferably, the concentration of collagenase I used according to the invention is between 0.5 to 10 mg/ml, even more preferably between 1-7 mg/ml, and most preferably between 2 and 4 mg/ml.

Such agents are readily available from commercial suppliers. Alternatively, agents such as trypsin, pepsin, proteases in combination with a divalent cation chelator eg. ethylenediaminetetraacetic acid (EDTA) can also be used. Other suitable agents will be familiar to persons skilled in the art.

Generally, the step of treating the bone fragments with a composition as described above is accompanied by mechanical agitation which assists in increasing the efficiency of the treatment process.

The washing of bone fragments and recovery/harvesting of cells according to the methods of the invention is preferably carried out in the presence of a buffer such as PBS. The cells are then preferably filtered through a filter unit such as a 40 μm filter.

After the cells are collected they may be concentrated by centrifugation, re-suspending the cells, filtering as described above, and pooling. However, it would be readily apparent to one skilled in the art upon reading the present disclosure that this can be done in any order, for example, the cells may be pooled prior to centrifugation and re-suspending and then filtered.

The bone marrow cells thus harvested according to the invention can then be further processed to isolate selected hematopoietic subsets according to standard methods.

Where it is intended to optimise harvest of cells of the HSC and/or progenitor cell lineage, it may be desirable to initially treat a non-human mammal with an agent which can be used to eliminate proliferating cells. Alternatively, agents such as 4-hydroperoxycyclophosphamide (4-HC), hydroxyurea or 5-fluorouracil (5-FU) can be utilised to eliminate cycling cells. These agents become incorporated into the DNA of cycling cells, which then undergo apoptosis. Due to their slow cycling, stem cells are spared from being destroyed by these agents.

In a third aspect, the present invention provides a method for isolating hematopoietic stem cells and/or progenitor cells from bone(s) harvested from a non-human mammal, the method comprising the steps of:
(a) administering to at the least one mammal an agent used to eliminate proliferating cells;
(b) obtaining a bone(s) containing bone marrow from the mammal;
(c) fragmentation of the bone(s);
(d) washing the bone fragments produced from step (c) and recovery of dislodged cells;
(e) treating the bone fragments with a composition to release cells adhered to the endosteal region of the bone; and
(f) harvesting the cells following treatment of the bone fragments according to step (e); and
(g) optionally combining the cells from steps (d) and (f).

The method according to this aspect may optionally comprise the further step of flushing the cavity of the bone to remove central bone marrow. This central bone marrow may be retained and then pooled with bone marrow cells released following treatment of the bone fragments according to step (e).

In a specific embodiment according to this aspect, the non-human mammal is a mouse.

Exemplary agents for this include, but are not limited to, 4-hydroperoxycyclophosphamide (4-HC), hydroxyurea and 5-fluorouracil (5-FU). The concentration of agent will depend on the size, physiology and pharmacological profile of the mammal, and it will be within the skill of one skilled in the art to optimize the dosage and harvest timing based on available information in the art and this disclosure. For example, where the agent is 5-FU, it is generally administered to a mouse at a concentration of 150 mg/kg, four days prior to harvesting the bone marrow.

The present invention also provides a kit for improving the isolation of hematopoietic stem cells and/or progenitor cells from bone marrow. The kit comprises an enzymatic reagent and one or more reagents for harvesting, washing and/or suspending the bone marrow cells. The kit will also comprise instructions to aid the user in the isolation methodology.

In a fourth aspect, the present invention provides a kit for isolating hematopoietic stem cells and/or progenitor cells from bone(s) harvested from a mammal, the kit comprising:
(a) a composition to release cells adhered to the endosteal region of the bone;
(b) one or more reagents for harvesting, washing and suspending the cells; and
(c) instructions for use of the reagents.

Preferably, the composition comprises a collagenase and optionally an agent selected from the group consisting of a protease, glycosidase and chelating agent and combinations thereof.

Preferably, the protease is dispase.

Preferably, the glycosidase is hyaluronidase.

Preferably, the chelating agent is ethylenediaminetetraacetic acid (EDTA).

Most preferably, the composition comprises a combination of 4 mg/ml dispase II and 3 mg/ml collagenase I.

Preferably, the reagent for harvesting, washing and suspending the bone marrow cells is phosphate buffered saline supplemented with 2% heat inactivated serum. More preferably, the heat inactivated serum is fetal calf serum.

The kit also optionally comprises a vessel for containing the cells such as a centrifuge tube. It may be necessary to employ more than a single centrifuge tube depending on the volume in which the cells are suspended prior to concentrating.

Optionally, the kit also comprises a device for fragmenting bone. An exemplary device for this is a mortar and pestle.

The kit also optionally contains a filter for separating cells from bone fragments. An exemplary device is a 40 μm filter.

A sub-population of cells comprising HSCs and/or progenitor cells can be isolated from the bone marrow cells harvested according to the invention by methods known to persons skilled in the art. For example, monoclonal antibodies can be used to identify markers. The selection process may be positive selection ie. selection for cells bearing markers characteristic of stem cells, or alternatively negative selection where selection is based on removal of cells bearing markers characteristic of differentiated or lineage committed cells. The antibodies may be attached to a solid support to allow for separation. Procedures for separation may include magnetic separation, using antibody coated magnetic beads, affinity chromatography, cytotoxic agents joined to a monoclonal antibody or used in conjunction with a monoclonal antibody, and panning.

One of the most effective ways of obtaining an enriched population of HSCs and/or progenitor cells is by fluorescence activated cell sorting (FACS) where multicolour analysis is used to select for cells on the basis of positive staining for Sca-1 and c-Kit and intermediate staining for the Thy-1 differentiation antigen. Typically, the method will also include an agent such as propidium iodide (PI) which stains non viable cells.

Once stem cells have been isolated, they may be propagated by growing in conditioned medium from stromal cells, or in medium comprising growth factors known to support the maintenance or differentiation of stem cells.

The cells may be used to reconstitute a lethally irradiated non-human recipient to study their ability to successfully restore hematopoiesis in the recipient. Alternatively, the cells may be used in transplantation therapy of human patients who have undergone chemo or myeloablative therapy.

Alternatively the cells can be used as a source of specific lineages, by providing for their maturation, proliferation and differentiation into one or more selected lineages by employing a variety of factor such as erythropoietin, GM-CSF, G-CSF, M-CSF, interleukins eg. IL-1, IL-2 etc, or stromal cells associated with the stem cells becoming committed to a particular lineage, or with their proliferation, maturation and differentiation.

The stem cells may also be used in the isolation and evaluation of factors associated with the differentiation and maturation of hematopoietic stem cells. Thus, the stem cells may be used for example, in assays to determine the activity of media such as conditioned media, evaluate fluids for cell growth activity, and involvement with dedication to particular lineages.

The stem cells may also be used to study the process and mechanisms responsible for bone marrow trans-marrow migration, lodgement and regulation within the HSC niche.

In order that the nature of the present invention may be more clearly understood, preferred forms thereof will now be described with reference to the following non-limiting examples.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Murine Isolation Method

Materials and Methods

Collagenase/Sispase

Weigh Dispase II to 4 mg/ml and shake into straight PBS. Weigh collagenase Type I to 3 mg/ml and dissolve into Dispase II solution. Make fresh each day.

LSK (Lin⁻ Sca⁺ Kit⁺) Isolation

The method for the isolation of cells located near the endosteal region of the bone in mice is described below:

1) Endosteal

a) Harvest femurs, tibias and iliac crests and remove all muscle.
b) Flush central marrow out using a 23 gauge or 26 gauge needle and 1 ml PBS-2% FCS (femurs and tibias versus iliac crests respectively) and discard flushed marrow.
c) Grind bones in mortar and pestle in PBS 2% Heat inactivated serum.
d) Wash bone fragments several times and filter cells through 40 μm filter, collecting a total of 200 ml for 10 mice.
e) Place bone fragments in 5 ml of 4 mg/ml Dispase II, 3 mg/ml Collagenase I in PBS and place at 37° C. shaking at 250 rpm for 5 min for up to 10 mice.
f) Wash fragments well with PBS by vigorously shaking and filtering through a 40 μm filter, collecting 100 ml total.
g) Centrifuge cells from step d and f at 400 rpm for 5 min at 4° C., filter through a 40 μm filter and pool.
h) Process cells as normal for isolation of selected hemopoietic subsets.

The method for the isolation of cells located within the central marrow region of the bone in mice is described below:

2) Central

a) Harvest femurs, tibias and iliac crests and remove all muscle.
b) Flush central marrow out using a 23 gauge or 26 gauge needle and 1 ml PBS-2% FCS (femurs and tibias versus iliac crests respectively) and discard bones.
c) Centrifuge cells at 400 rpm for 5 min at 4° C., filter through a 40 μm filter and pool.
d) Process cells as normal for isolation of selected hematopoietic subsets.

The method for the isolation of all bone marrow cells in mice is described below:

3) Enhanced

a) Harvest femurs, tibias and iliac crests and remove all muscle.
b) Grind bones in mortar and pestle in PBS 2% Heat inactivated serum.
c) Wash bone fragments several times and filter cells through 40 μm filter, collecting a total of 200 ml for 10 mice.
d) Place bone fragments in 5 ml of 4 mg/ml Dispase II, 3 mg/ml Collagenase I in PBS and place at 37° C. shaking at 250 rpm for 5 min for up to 10 mice.
e) Wash fragments well with PBS by vigorously shaking and filtering through a 40 μm filter, collecting 100 ml total.
f) Cnetrifuge cells from step c and e at 400 rpm for 5 min at 4° C., filter through a 40 μm filter and pool.
g) Process cells as normal for isolation of selected hemopoietic subsets
   LSK Separation from Whole Bone Marrow

Low density cells (<1.077 g/cm3) were isolated by discontinuous density centrifugation using Nycoprep for animals (Accurate Chemical and Scientific Corporation, Westbury, N.Y.) and lin−cells separated as previously described (Nilsson et al., Blood, 2001). Briefly, cells were labelled with a cocktail of biotinylated rat anti-mouse antibodies: B220, Mac-1, Gr-1, CD4, CD8, CD3, CD5 and Ter119. Lin+ cells were removed by immunomagnetic selection using MACS (Miltenyi Biotec, Bergisch, Gladbach, Germany). Lin−cells were labelled with Sca-1-FITC (Pharmingen; 1 μg/5×10⁶ cells), c-kit-PE (Pharmingen; 1 μg/5×10⁶ cells), and strepavidin-Red 670 (Gibco, Grand Island, N.Y.; 1/160 final concentration). For long-term transplant analysis, purified rat anti-mouse antibodies were used in combination with Sca-biotin, c-kit-APC and strepavidin-Red 670. LSK cells were isolated by fluorescence-activated cell sorting (FACS).

LPP-CFC and HPP-CFC Assays

Low and high proliferative potential colony forming cells (LPP-CFC and HPP-CFC respectively) were assayed in a double layer nutrient agar culture system as previously described (Bartelmez S H, Bradley T R, Bertoncello I et al, Interleukin 1 plus interleukin 3 plus colony-stimulating factor 1 are essential for clonal proliferation of primitive myeloid bone marrow cells. Exp Hematol, 1989; 17:240-245), except that SCF was added to CSF-1, IL-1 and IL-3 to analyze HPP-CFC.

LSK Culture

Sorted LSK were cultured in 100 μl Cellgro media supplemented with rat SCF (75 ng/ml), and human FLT3 Ligand (FLT3-L) (50 ng/ml), IL-6 (10 ng/ml) and IL-11 (10 ng/ml) per well in a 96 well plate. All cells were cultured at 37° C. in 5% O2, 10% CO2 and 85% N2. Cells were grown for 6 days prior to counting.

5- (and-6)-carboxyfluorescein Diacetate Succinimidyl Ester (CFSE) Labeling.

Cells to be transplanted for spatial distribution analysis were labeled with the fluorescent dye CFSE (Molecular Probes, Eugene, Oreg.) as previously described (Nordon et al., High resolution cell division tracking demonstrates the Flt3-ligand-dependence of human marrow CD34+CD38− cell production in vitro. Brit. J. Haematol., 1997; 98:528-539. Briefly, cell populations were resuspended in PBS 0.5% HI FCS at a density of 106 cells/mL and pre incubated at 37° C. for 3 minutes. CFSE was diluted to 5 mM in dimethyl sulfoxide and then to 5 μM in PBS. CFSE was added to the cells to give a final concentration of 0.5 μM, and the dye solution/cell mixture incubated at 37° C. for a further 10 minutes. Staining was stopped by adding 10 times the dye solution/cell volume of ice-cold PBS containing 20% FCS.

Analysis of Cell Homing and Spatial Distribution.

The homing ability and spatial distribution of CFSE positive cells was analyzed 15 hrs post-transplant. For the analysis of homing, endosteal and central marrow samples were prepared from individual mice as described above. In addition, single cell suspensions were made from each spleen. Marrow cells were labeled with CD45-pe and spleen cells with B220-pe. Analysis of the proportion of CFSE+ donor cells in each fraction was determined using CD45-pe and B220-pe as the denominator for the total number of cells analyzed by flow cytometry for marrow and spleen respectively. Preparation of samples for spatial distribution was as previously described (Nilsson S K, Johnston H M, Coverdale J A. Spatial localization of transplanted hemopoietic stem cells: inferences for the localization of stem cell niches. Blood, 2001; 97:2293-2299). The location of CFSE labeled cells (positive cells) from at least 6 longitudinal sections per transplant recipient was recorded. Central longitudinal sections were analyzed as opposed to transverse sections, as each individual section encompasses more of the entire femur. To ensure that individual cells were only analyzed once, every alternate 3.5 μm section was analyzed. The location of positive cells was designated as either endosteal (previously arbitrarily defined as within 12 cells of the endosteum) or central (greater than 12 cells from either endosteum).

LSK Transplantation

The ability of populations of mice strain PTPRCA and C57 male donor LSK cells to reconstitute hemopoiesis long-term was analyzed in C57 female recipients receiving a near-lethal dose of irradiation (9.5 Gy) in two equal fractions separated by a 4 hr interval, delivered from 2 opposing 137Cs sources (Gammacell 40; Atomic Energy of Canada, Ottawa, Canada) at a dose-rate of 1.4 Gy/min.

Long-Term Transplant Analysis.

Bone marrow was isolated from individual transplant recipients 12 weeks post-transplant using the enhanced method described above. Both whole marrow and LSK cells (isolated as described above) were labelled with Ly5.1-pe (PTPRCA) and Ly5.2-fitc (C57) to determine the proportion of PTPRCA LSK contribution and all Ly5.2+ cells sorted. The proportion of male C57 LSK cell donor contribution was determined from the sorted Ly5.2+ fraction using the “hot-shot” DNA isolation method detailed below and real time PCR for Y chromosome. The proportion of donor cells was then mathematically calculated.

“Hot Shot” DNA Isolation.

Cells were dry pelleted and re suspended in 50 mM NaOH (600 μl for cell numbers greater than 100 000 and 300 μl for less). Heat the cells for 10 minutes at 90° C. Vortex quickly and add 1M Tris, pH 8 (100 μl for cell numbers greater than 100 000 and 50 μl for less). Centrifuge at 13000 rpm for 10 minutes and transfer supernatant to a new tube.

Results

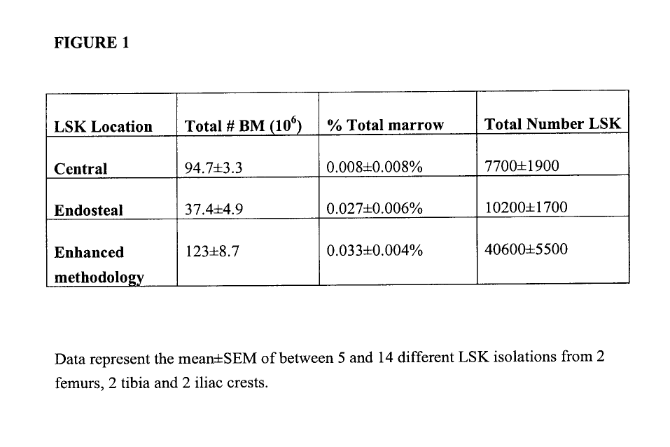

Bone marrow cells were obtained by either flushing the bone marrow from the bone according to prior art methods eg. by using a syringe, or obtained by the method according to the present invention. The proportion of LSK cells within the starting bone marrow (SBM) and lineage negative (Lin−) fractions as well as the total number of LSK isolated using the three separative strategies. Values are the mean+std error of the mean (SEM) of at least 8 individual groups of animals are presented in FIG. 1.

There was a significant difference in the total number of LSK cells isolated using the enhanced isolation separative strategy.

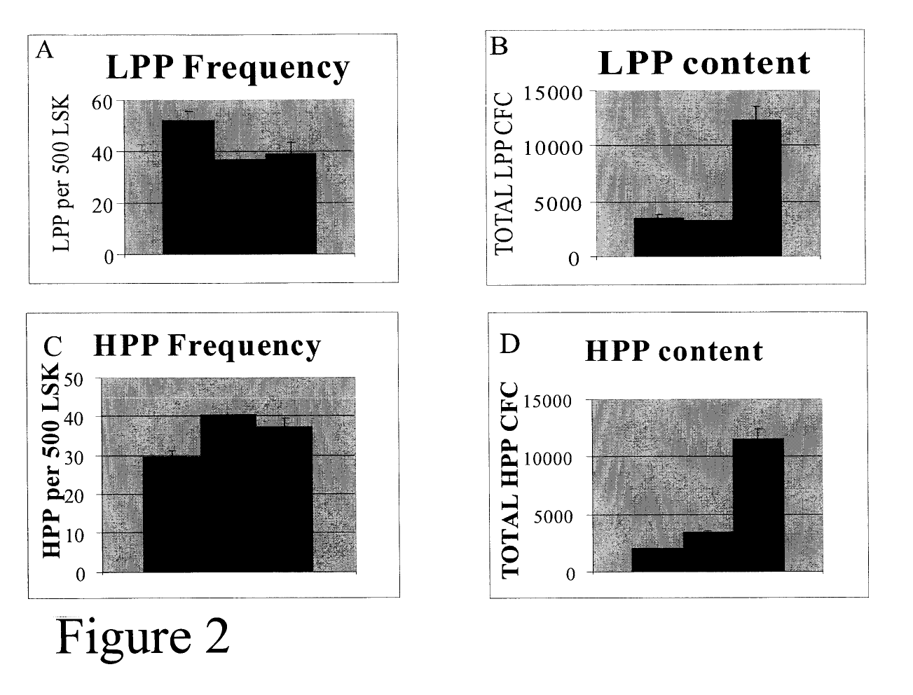

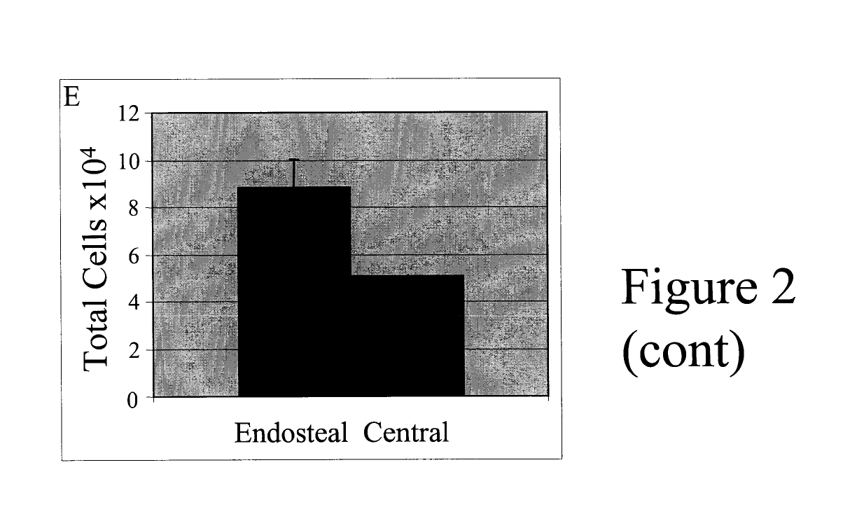

FIG. 2 shows an analysis of the frequency and content of low-proliferative-potential colony-forming-cells (LPP-CFC) (A and B respectively). There was a significant increase in the proportion of mature progenitors or LPP-CFC in the central (flushed) versus endosteal (ground) or enhanced (norm) fractions (p<0.01). In addition, using the enhanced method, there was a significant increase in the total number of LPP-CFC recovered. Conversely, analysis of the frequency and content of high-proliferative-potential colony-forming-cells (HPP-CFC) (C and D respectively) demonstrated a significant decrease in the proportion and total number of immature progenitors of HPP-CFC in the central (flushed) versus endosteal (ground) or enhanced (norm) fractions (p<0.002 and p<0.001 respectively). Furthermore, LSK cells isolated from the endosteal (gr) region had a significantly higher proliferative potential after 6 days culture in Cellgro plus 4 factor in vitro, compared to those isolated from the central (fl) region (E) (p<0.001). The Lin-Sca-1+Kit+(LSK) were isolated from the bone marrow cells by MACS (to remove lineage positive and leave lineage negative cells) and FACS (for isolation of highly enriched HSC sub-populations) and 100 cells seeded into a culture dish containing Cellgro and four growth factors. After day 6, the total number of cells generated from the LSK cells were ascertained and the results are presented in FIG. 1.

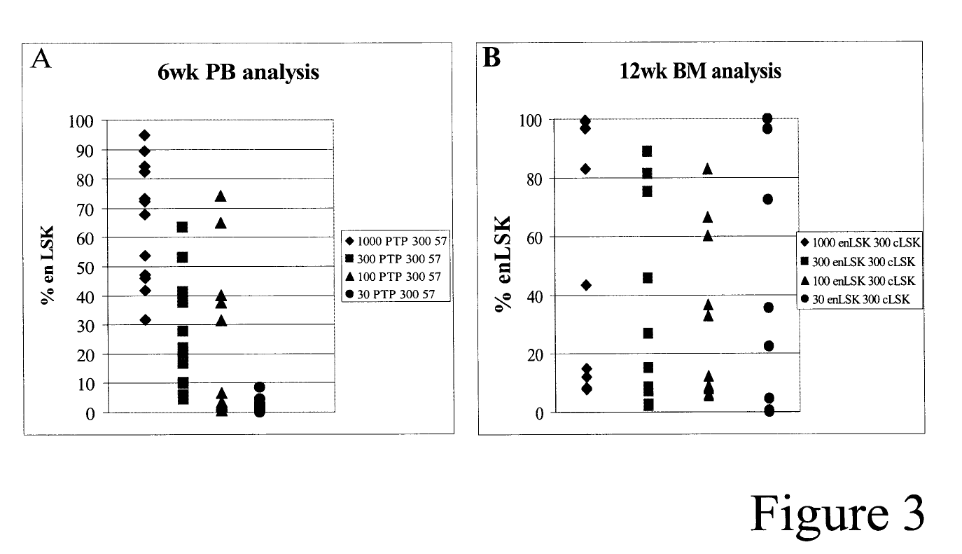

FIG. 3 shows an analysis of whole bone marrow (BM) (A) following the competitive transplant of various numbers of LSK isolated from the endosteal region (PTP) versus central region (57). There was no significant difference between the detected and expected contribution to hemopoietic reconstitution (43±14%, 31±12%, 34±10%, 4±3% compared to the expected 77%, 50%, 25%, 9% respectively) when 1000, 300, 100 or 30 LSK isolated from the endosteal region were competed with 300 LSK isolated from the central region. Furthermore, analysis of LSK (B) from the same recipient animals also demonstrated no significant difference between the detected and expected contribution to hemopoietic reconstitution (59±14%, 27±11%, 62±16%, 0±0% compared to the expected 77%, 50%, 25%, 9% respectively) following the same competitive transplant. This suggests that LSK cells isolated from both the endosteal and central marrow regions contribute equally to the reconstitution of all levels of the stem cell hierarchy in this in vivo transplant model.

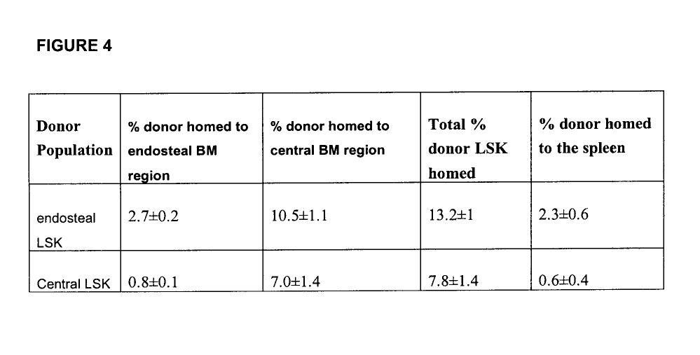

Transplanted LSK isolated from the endosteal region showed a significantly greater ability to home to the BM and specifically the endosteal region compared to those isolated from the central region (p<0.007 and p<0.0002 respectively) as demonstrated in FIG. 4. Furthermore, transplanted LSK isolated from the endosteal region showed a significantly greater ability to home to the spleen compared to those isolated from the central region (p<0.007 and p<0.0002 respectively).

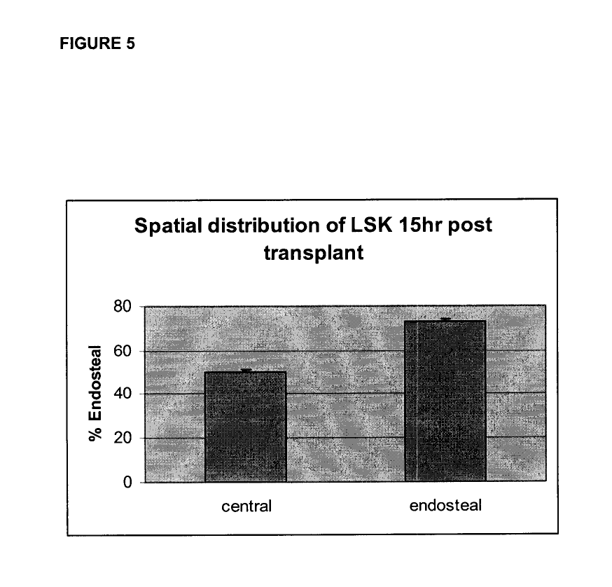

The spatial distribution of CFSE+ LSK 15 hr post-transplant demonstrated a significant increase in the proportion of LSK isolated from the endosteal region located within this region 15 hr post-transplant compared to LSK isolated from the central marrow region (p<0.0001) FIG. 5.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method for isolating cells from bone(s) harvested from a mammal, the method comprising the steps of:
(a) obtaining a bone(s) containing bone marrow from the mammal;
(b) fragmentation of the bone(s);
(c) washing the bone fragments produced from step (b) and recovery of dislodged cells;
(d) treating the bone fragments with a composition to release cells adhered to the endosteal region of the bone;
(e) harvesting the cells following treatment of the bone fragments according to step (d); and
(f) optionally combining the cells from steps (c) and (e).

2. A method for isolating cells from bone(s) harvested from a mammal, the method comprising the steps of:
(a) obtaining a bone(s) containing bone marrow from the mammal;
(b) flushing the cavity of the bone to remove central bone marrow;
(c) fragmentation of the bone(s);
(d) washing the bone fragments produced from step (c) and recovery of dislodged cells;
(e) treating the bone fragments with a composition to release cells adhered to the endosteal region of the bone;
(f) harvesting the cells following treatment of the bone fragments according to step (e); and
(g) optionally combining the cells from steps (d) and (f).

3. A method according to claim 1 or 2 wherein the cells are hematopoietic stem and/or progenitor cells.

4. A method according to claim 1 or 2 wherein the cells are malignant cells.

5. A method according to claim 1 wherein the mammal is a mouse.

6. A method according to claim 1 wherein the mammal is a human.

7. A method according to claim 5 wherein the bone(s) are selected from one or more of the group consisting of femur, tibia and iliac crest.

8. A method according to claim 6 wherein the bone(s) are selected from one or more of the group consisting of spine, rib, sternum, femur and iliac crest.

9. A method according to claim 1 wherein the fragmentation results in a substantial proportion of the bone consisting of fragments of a size range of from about 40 microns to about 1000 microns.

10. A method according to claim 1 wherein the composition which releases cells adhered to the endosteal region of the bone comprises a collagenase and optionally an agent selected from the group consisting of a protease, a glycosidase and chelating agent and combinations thereof.

11. A method according to claim 10 wherein the protease is dispase.

12. A method according to claim 10 wherein the glycosidase is hyaluronidase.

13. A method according to claim 10 wherein the chelating agent is ethylenediaminetetraacetic acid (EDTA).

14. A method according to claim 10 wherein the composition comprises a combination of 4 mg/ml dispase II and 3 mg/ml collagenase I.

15. A method according to claim 11 wherein the the concentration of Dispase II is between 0.5 to 10 mg/ml, even more preferably between 2-8 mg/ml, and most preferably between 3 and 5 mg/ml.

16. A method according to claim 10 wherein the concentration of collagenase I used according to the invention is between 0.5 to 10 mg/ml, even more preferably between 1-7 mg/ml, and most preferably between 2 and 4 mg/ml.

17. A method for isolating hematopoietic stem cells and/or progenitor cells from bone(s) harvested from a non-human mammal, the method comprising the steps of:
(a) administering to at the non-human mammal an agent used to eliminate proliferating cells;
(b) obtaining a bone(s) containing bone marrow from the mammal;
(c) fragmentation of the bone(s);
(d) washing the bone fragments produced from step (c) and recovery of dislodged cells;
(e) treating the bone fragments with a composition to release cells adhered to the endosteal region of the bone; and
(f) harvesting the cells following treatment of the bone fragments according to step (e); and
(g) optionally combining the cells from steps (d) and (f).

18. A method according to claim 17 wherein the method further comprises the step of flushing the cavity of the bone to remove central bone marrow.

19. A method according to claim 17 wherein the non-human mammal is a mouse.

20. A method according to claim 17 wherein the agent used to eliminate proliferating cells is selected from the group consisting of 4-hydroperoxycyclophosphamide (4-HC), hydroxyurea and 5-fluorouracil (5-FU).

21. A kit for isolating hematopoietic stem cells and/or progenitor cells from bone(s) harvested from a mammal, the kit comprising:
(a) a composition to release cells adhered to the endosteal region of the bone;
(b) one or more reagents for harvesting, washing and suspending the cells; and
(c) instructions for use of the reagents.

22. A kit according to claim 21 wherein the composition comprises a collagenase and optionally an agent selected from the group consisting of a protease, glycosidase and chelating agent and combinations thereof.

23. A kit according to claim 22 wherein the protease is dispase.

24. A kit according to claim 22 wherein the glycosidase is hyaluronidase.

25. A kit according to claim 22 wherein the chelating agent is ethylenediaminetetraacetic acid (EDTA).

26. A kit according to claim 21 wherein the composition comprises a combination of 4 mg/ml dispase II and 3 mg/ml collagenase I.

27. A kit according to claim 21 wherein the reagent for harvesting, washing and suspending the bone marrow cells is phosphate buffered saline supplemented with 2% heat inactivated serum, preferably fetal calf serum.

28. A kit according to claim 21 wherein the kit further comprises a device for fragmenting bone.

29. A kit according to claim 21 wherein the kit further comprises a filter for separating cells from bone fragments.
